(19) United States
(12) Patent Application Publication
(10) Pub. No.: US 2025/0034620 A1
(43) Pub. Date: Jan. 30, 2025

(54) PROBE FOR MONITORING RNA SPLICING AND METHODS THEREOF

(71) Applicant: Yale University, New Haven, CT (US)

(72) Inventors: Anna Marie Pyle, New Haven, CT (US); Olga Fedorova, New Haven, CT (US); Qusay Omran, New Haven, CT (US)

(21) Appl. No.: 18/835,094

(22) PCT Filed: Feb. 3, 2023
(86) PCT No.: PCT/US2023/061958

§ 371 Date: Aug. 1, 2024

Related U.S. Application Data
(60) Provisional application No. 63/306,796, filed on Feb. 4, 2022.

(51) Int. Cl. C12Q 1/6818; C12Q 1/6895

(52) U.S. Cl. CPC C12Q 1/6818; C12Q 1/6895

(57) ABSTRACT

Described herein is a probe for monitoring specific RNA splicing events, with utility for identifying molecular modulators of RNA splicing. The probe includes a nucleic acid, a first fluorescent element attached to a first end of the nucleic acid, and a second fluorescent element attached to a second end of the nucleic acid. The fluorescence signal of the probe changes when the probe hybridizes with either a pre-splicing RNA molecule or a splicing product. Also described herein is a method of monitoring RNA splicing and a method of screening of modulators of RNA splicing using the probe.

### CROSS-REFERENCE TO RELATED APPLICATIONS

The present application claims priority under 35 U.S.C. § 119 (e) to U.S. Provisional Patent Application No. 63/306,796, filed Feb. 4, 2022, which is incorporated herein by reference in its entirety.

### SEQUENCE LISTING

The ASCII text file named “047162-7348WO1 (01870) _Sequence Listing” created on Jan. 27, 2023, comprising 5.95 Kbytes, is hereby incorporated by reference in its entirety.

### BACKGROUND

Group II introns are a class of autocatalytic RNA ribozymes found in bacteria and the organellar genomes of fungi, plants, and protists. The self-splicing activity of group II introns is likely an ancient precursor to the eukaryotic spliceosome, which shares the characteristic two-step sequential mechanism by which group II introns self-splice. The distinctive structural features of group II introns include conserved six domains and overall secondary structure despite the lack of phylogenetic conservation at the sequence level. Because of the structured nature and ubiquity in mitochondrial genes responsible for the respiration of bacteria and fungi, group II introns represent a promising therapeutic target that have largely been neglected by conventional screening and the development of new drug discovery platforms.

Misregulation of alternative splicing causes harmful diseases. It has been reported that approximately 300 genes and 370 diseases are linked to disruption of alternative splicing. For example, myotonic dystrophy is a disease associated with alternative splicing. Genetic mutations affecting splicing have been linked to ALS and retinitis pigmentosa. In cancer, alternative splicing generates tumor-associated protein isoforms that promote tumorigenesis. For example, hypoxia-induced alternative splicing is one of the major driving forces in tumor development. Modulators of RNA splicing have been tested as anti-cancer agents. Further, group I and group II introns are found in the genome of many organisms, some of them pathogenic as pathogenic fungi. Thus, small molecule inhibitors of splicing events specific for eukaryotic pathogens could become a promising new class of antifungal drugs. However, methods of directly monitoring individual RNA splicing events are lacking. In particular, there are currently no robust fluorescence-based probes or other high-throughput methods to directly monitor splicing of group I introns, group II introns or nuclear pre-mRNA introns in metazoans. This hinders development of assays to search for modulators of specific splicing events.

Therefore, there is a need to develop compositions and methods for directly monitoring individual, sequence-specific RNA splicing events using high-throughput methods such as fluorescence-based probes and assays. The instant invention addresses this need.

### SUMMARY

In some aspects, the present invention is directed to the following non-limiting embodiments:

### Probe

In some aspects, the present invention is directed to a probe, such as a probe for monitoring the sequence-specific splicing of individual RNA exons.

In some embodiments, the probe includes a nucleic acid, a first fluorescent element attached to a first end of the nucleic acid, and a second fluorescent element attached to a second end of the nucleic acid.

In some embodiments, the nucleic acid includes: a first stem portion; a second stem portion; and a loop portion.

In some embodiments, the loop portion of the nucleic acid does not hybridize to a pre-splicing RNA molecule and hybridizes to a splicing product of the RNA molecule.

In some embodiments, the loop portion of the nucleic acid hybridizes to the pre-splicing RNA molecule and does not hybridize to the splicing product of the RNA molecule.

In some embodiments, the first stem portion and the second stem portion are at least practically complementary to each other and can form a stem structure when the loop portion is not hybridized to the pre-splicing RNA molecule or the splicing product.

In some embodiments, the fluorescent signal of the probe when the loop portion of the nucleic acid hybridizes to the pre-splicing RNA molecule or the splicing product, and the fluorescent signal of the probe when the loop portion of the nucleic acid does not hybridize to the pre-splicing RNA molecule or the splicing product, are different.

In some embodiments, the nucleic acid is RNA, DNA, or a modified nucleic acid.

In some embodiments, when the loop portion of the nucleic acid is not hybridized to the pre-splicing RNA molecule or the splicing product, the first fluorescent element and the second fluorescent element are in proximity to each other such that a fluorescent property of the first fluorescent element is affected by the second fluorescent element.

In some embodiments, the first fluorescent element is a fluorophore and the second fluorescent element is a quencher that decreases the fluorescence intensity of the fluorophore.

In some embodiments, the first fluorescent element is a donor in a fluorescence resonance energy transfer (FRET) pair, and the second fluorescent element is an acceptor in the FRET pair.

In some embodiments, the loop portion hybridizes to a segment of the splicing product of the RNA molecule, and the segment of the splicing product spans at least two consecutive exons.

In some embodiments, the loop portion hybridizes to a segment of a lariat intronic RNA formed by the splicing, and the segment of the lariat intronic RNA comprises the point where the 5′-end and the branchpoint adenosine residue of the intron join during the splicing process.

In some embodiments, the loop portion hybridizes to a segment of the pre-splicing RNA molecule, and the segment of the pre-splicing RNA molecule spans at least one exon and at least one intron adjacent to the at least one exon.

In some embodiments, the length of the first stem portion or the length of the second stem portion ranges from 3 nucleotides to 10 nucleotides.

In some embodiments, in the loop portion, the number of nucleotides complementary to the pre-splicing RNA molecule or the splicing product ranges from 10 to 30.

In some embodiments, the melting temperature of a hybridization product formed by the probe and the pre-splicing RNA molecule or the splicing product ranges from 40° C. to 85° C.

In some embodiments, the nucleic acid further includes a linker. In some embodiments, the linker is at the first end of the nucleic acid, at the second end of the nucleic acid, between the first stem portion and the loop portion, and/or between the loop portion and the second stem portion.

In some embodiments, the length of the linker ranges from 1 nucleotide to 15 nucleotides.

### Method of Monitoring RNA Splicing

In some embodiments, the present invention is directed to a method of monitoring RNA splicing.

In some embodiments, the method comprises preparing a mixture including an unspliced RNA molecule; and a probe.

In some embodiments, the probe is the same as or similar to those described elsewhere herein.

In some embodiments, a change of a fluorescence signal of the probe indicates the sequence-specific splicing of the RNA molecule.

In some embodiments, the amount of fluorescence signal change is proportional to the efficiency of an individual RNA splicing event.

In some embodiments, the level of the fluorescence signal of the probe corresponds to the amount of the unspliced precursor RNA molecule or the level of a splicing product that derives from that precursor RNA molecule.

In some embodiments, the sequence-specific splicing of the RNA molecule is specific to a developmental process or a differentiated tissue in a subject.

In some embodiments, the sequence-specific splicing of the RNA molecule is present in a subject, tissue or cell having a disease or disorder, and the sequence-specific splicing of the RNA molecule is not present in a corresponding healthy subject, tissue or cell.

In some embodiments, the disease or disorder includes at least one selected from the group consisting of a neurodegenerative disease, a developmental disorder, a cardiac disorder, an autoimmune disease, a cancer, and an infectious disease.

### Method of Screening Modulators of RNA Splicing

In some aspects, the present invention is directed to a method of screening modulators of RNA splicing.

In some embodiments, the method includes: preparing a first mixture including: an unspliced precursor RNA molecule and a probe; determining a first fluorescence signal level change of the first mixture; preparing a test mixture including the unspliced precursor RNA molecule, the probe, and a test compound; determining a second fluorescence signal level change of the second mixture.

In some embodiments, the probe is the same as or similar to those as detailed elsewhere herein.

In some embodiments, a difference between the first fluorescence signal level change and the second fluorescence signal level change indicates that the test compound is a modulator of the sequence-specific splicing of the RNA molecule.

In some embodiments, the method is a method of screening compounds for treating, ameliorating or preventing a disease or a disorder, and the sequence-specific splicing of the RNA molecule is a cause or a contributing factor to the disease or the disorder.

In some embodiments, the disease or the disorder includes at least one selected from the group consisting of a neurodegenerative disease, a developmental disorder, a cardiac disorder, an autoimmune disease, a cancer or an infectious disease.

In some embodiments, the method is a method of screening anti-pathogenic compounds, and the splicing of the RNA molecule is specific to a eukaryotic pathogen and does not take place in the host of the eukaryotic pathogen.

In some embodiments, the method further includes applying a gene editing technology that affects an amount and/or sequence of the unspliced RNA molecule or a splicing product thereof.

In some embodiments, the gene editing technology comprises at least one selected from the group consisting of an RNA interference technology, a CRISPR-Cas12 system, and a CRISPR-Cas13 system.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrates a sequential branch-point self-splicing reaction observed in group II introns in vitro, in accordance with some embodiments. As shown in FIG. 1, although group II introns are not phylogenetically conserved at the sequence level, they are defined in the first step by nucleophilic at-tack by the 2′-hydroxyl of a conserved “bulged” adenosine residue at the 5′ splice site. The liberated 5′-exon then attacks the 3′ splice site to form the ligated spliced exons product in the second step, producing the lariat product as well. Evolution of a linear intron is sometimes observed in group II intron self-splicing reactions, usually by means of hydrolytic opening of the lariat product or an alternative hydrolytic splicing process not featured in this figure.

FIG. 2 is a schematic of molecular beacon hybridization to a target sequence, in accordance with some embodiments. As shown in FIG. 2, in the stem-loop form, the molecular beacon's fluorophore and quencher are forced into proximity with each other, limiting fluorescence activity. Upon recognition of the target sequence complementary to the loop sequence, however, the beacon hybridizes to the target, distancing the fluorophore from the quencher so that fluorescence can serve as a signal of the target's presence in solution.

FIG. 3A: Recognition of the synthetic target RNA and the ligated exons formed during splicing by molecular beacons. Beacons were incubated with buffer-only (negative control, light grey bars), with the HC preRNA splicing reaction (dark grey bars)), or with the HCSE spliced exon oligonucleotide (positive control, striped bars). Buffer composition in each well reflects optimal reaction conditions. Data represent the average of n=3 independent experiments. Error bars are s.e.m. FIG. 3B: Comparison of the molecular beacon signal under different reaction conditions. 50 nM MB18 was incubated with either the synthetic HCSE oligo or the HC preRNA in either optimal reaction buffer (light grey bars), buffer lacking MgCl2 (striped bars), or in reaction buffer containing an equimolar amount of EDTA (dark grey bars). Data represent the average of n=3 independent experiments. Error bars are s.e.m.

FIGS. 4A-4B depict the progress of self-splicing reaction in parallel beacon and splicing gel assays, in accordance with some embodiments. FIG. 4A depicts representative 5% denaturing polyacrylamide splicing gel of the radiolabeled H.c.LSU RNA construct showing depletion of precursor and accumulation of reaction products, including spliced exons, over the course of the reaction. FIG. 4B depicts plots of RFUs from the beacon assay time course and fraction of radiolabeled spliced exons at each time point on gel. Reaction rates of 0.023±0.009 min⁻¹ from the beacon assay and 0.036±0.009 min⁻¹ from the splicing gel assay were derived. Data represent the average of n=3 independent experiments. Error bars are s.e.m.

FIG. 5 depicts the time courses of the H.c.LSU splicing reaction under optimal conditions and in 5% DMSO, in accordance with some embodiments. In FIG. 5, each reaction was initiated with the addition of 10 mM MgCl₂ and quenched at each timepoint with equimolar EDTA. The splicing buffer was composed of optimal reaction conditions (50 mM HEPES (pH 7.5), 150 mM NH₄Cl, and 10 mM MgCl₂). 5% DMSO was added in place of an equivalent volume of water for reactions with that condition. Rate constants of 0.043±0.013 min⁻¹ and 0.048±0.012 min⁻¹ were derived for the reactions with and without 5% DMSO, respectively. Data represent the average of n=2 independent experiments. Error bars are s.e.m.

FIG. 6 depicts the plot of rate constants (kobs) against mitoxantrone concentrations, in accordance with some embodiments. Each data point represents the rate determined by fitting a simple exponential curve to a time course of the self-splicing reaction in the presence of the respective inhibitor concentration. A logistic curve fit produced a K_{I} value of 2.24 μM±0.16.

FIG. 7 shows the predicted secondary structure of the H.c.LSU group II intron, in accordance with some embodiments. The conserved six domains observed for group II introns are labeled within the figure as D1-D6. The branchpoint adenosine in D6 is indicated by an arrow.

FIGS. 8A-8B depict representative time course data for the determination of the K_{I} of mitoxantrone, in accordance with some embodiments. FIG. 8A depicts the time course data in the presence of concentrations from 5 nM to 2 μM. FIG. 8B shows data for 1 μM to 300 μM. The 90 min time point data for FIG. 8A were outliers and were therefore excluded from rate determination.

FIGS. 9A-9D illustrate a probe for monitoring RNA splicing in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In the study described herein (“the present study”), a probe able to monitor sequence-specific RNA splicing was developed. Using a group II intron-containing mitochondrial LSU rRNA from Histoplasma capsulatum (H. capsulatum) as a non-limiting example, the present study demonstrated that the probe is both able to monitor splicing kinetics, and suitable for high throughput screening of modulators of the splicing process.

Accordingly, in some aspects, the present invention is directed to a probe for monitoring RNA splicing.

In some aspects, the present invention is directed to a method of monitoring RNA splicing.

In some aspects, the present invention is directed to a method of screening modulators that alters an RNA splicing process, which is required for the function of an organism.

It is worth noting that, although the present study uses the self-splicing of a group II intron-containing fungal rRNA as a non-limiting example, based on the disclosure of the instant specification, it would be apparent to one of ordinary skill in the art that the probe and methods thereof are applicable to all other splicing processes, as well, such as the formation of specific alternative splicing products in eukaryotic nuclei (e.g., during pre-mRNA splicing by the spliceosome).

It is also worth noting that the method of detecting the RNA splicing, such as detecting the specific pre-mRNA alternative splice variants, using the probe herein is sometimes combined with a DNA/RNA editing technology, such as any editing technology that affect the sequence and/or amount of an RNA molecule monitored by the instant probe. For example, the instant probe can be used in combination with CRISPR-Cas12, CRISPR-Cas13 and/or other RNA gene editing systems to provide an orthogonal cleavage readout for detection of splice products.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

“Hybridize” as used herein refers to two full complementary or partially complementary single-stranded DNA or RNA molecules form a single double-stranded molecule through base pairing.

“Fluorescent element” as used herein, refers to a fluorophore and/or a quencher.

“Fluorophore” as used herein refers to a fluorescent substance that can re-emit light upon light excitation. Fluorophores include, in non-limiting embodiments, organic small molecules having a molecular weight ranging from 20-1000 Dalton, fluorescent proteins, and inorganic optical probes such as quantum dots.

“Fluorescence resonance energy transfer,” “Forster resonance energy transfer,” or “FRET” as used herein refers to the phenomenon that an excited donor transfers energy (not an electron) to an acceptor group through a non-radiative process. FRET is highly distance-dependent, and the energy transfer efficiency decreases dramatically as the distance between the donor and the acceptor increases.

“Quencher” of a fluorophore, as used herein, refers to a substance capable of absorbing the light emitted from the fluorophore. Quenchers often re-emitting the energy absorbed as either heat (e.g., dark quenchers) or visible light (e.g., fluorescent quenchers). Some quenchers function through fluorescence resonance energy transfer, but other mechanisms of quenching exist as well and the instant specification is not limited thereto.

“Melting temperature” or “Tm” as used herein refers to the temperature at which half of the nucleic acid strands are in the random coil or single-stranded state. The melting temperature of a nucleic acid depends on a variety of factors, such as intrinsic factors including the type of the nucleic acid (DNA: DNA, DNA: RNA and RNA: RNA strands that have the same or similar nucleotide compositions most likely have different melting temperatures), the length of the nucleic acid (shorter nucleic acids often have lower melting temperatures), the nucleotide sequence composition (higher CG content often results in higher melting temperature), and extrinsic factors including salt concentration, salt type, concentrations of the nucleic acid strands, and the presence/absence of additional molecules that affects the interaction between two strands of nucleic acid molecules. Melting temperature of the annealing product of two nucleic acid strands can be experimentally determined, or be predicted with reasonably accuracy by known methods, such as those described in Kibbe et al. (Nucleic Acids Res. 2007 July; 35 (Web Server issue): W43-W46), Markham et al. (Nucleic Acids Res. 2005 Jul. 1; 33 (Web Server issue): W577-81), Ghosh et al. (PNAS Jun. 23, 2020 117 (25) 14194-14201), or Dimitrov et al. (Biophysical Journal, Volume 87, Issue 1, July 2004, Pages 215-226). The melting temperatures as described herein refer to the melting temperatures of 50 nm to 200 nm of nucleic acid strands in a solution of 50 mM HEPES (pH 7.5), 150 mM NH₄Cl, 10 mM MgCl₂, and water.

As used herein, when two fluorescent elements are said to be “in proximity to each other,” the distance between the two fluorescent elements allow one of the fluorescent element to exert about 25% or more, such as about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, of the maximal influence on the other fluorescent element.

As an example, the “maximal influence” a quencher on a fluorophore means the highest quenching effect achievable by the quencher. As another example, the “maximal influence” of a donor on an acceptor, or an acceptor on a donor in FRET is the maximal efficiency achievable by the donor/acceptor pair.

When two fluorescent elements are said to be “away from each other,” the influence of one of the fluorescent elements on the other fluorescent element decreases to about 75% or less, such as about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, the influence level when the two fluorescent elements are in proximity to each other.

As used herein, the term “disease” refers to a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

The term “diagnosis,” as used herein, refers to the determination of a condition of a subject, such as determining whether the subject has a particularly disease condition, susceptibility, or other trait.

### Probe for Monitoring RNA Splicing

The present study developed probes for monitoring sequence-specific, individual RNA splicing events, and further demonstrated that the probe is able to monitor the splicing kinetics and suitable for used in high throughput screenings.

Accordingly, in some aspects, the instant invention is directed to a probe for monitoring RNA splicing.

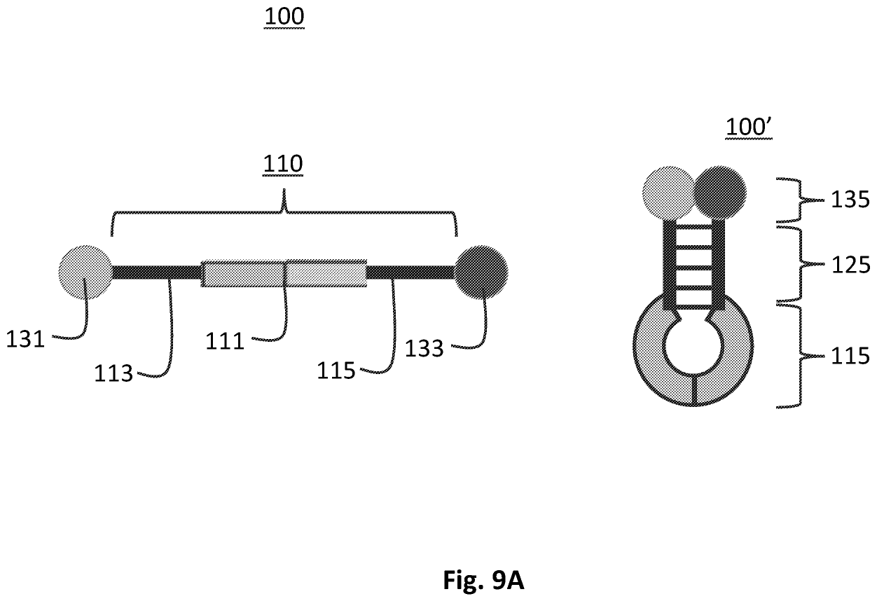

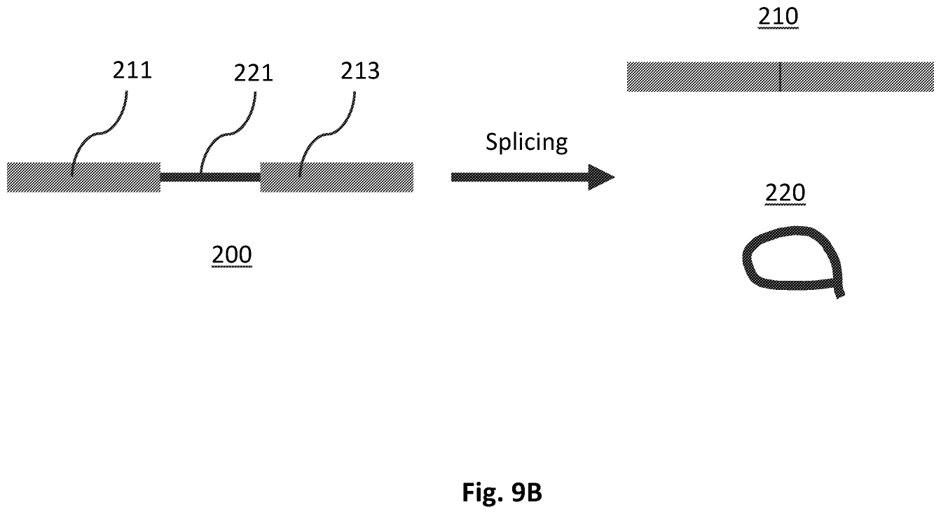

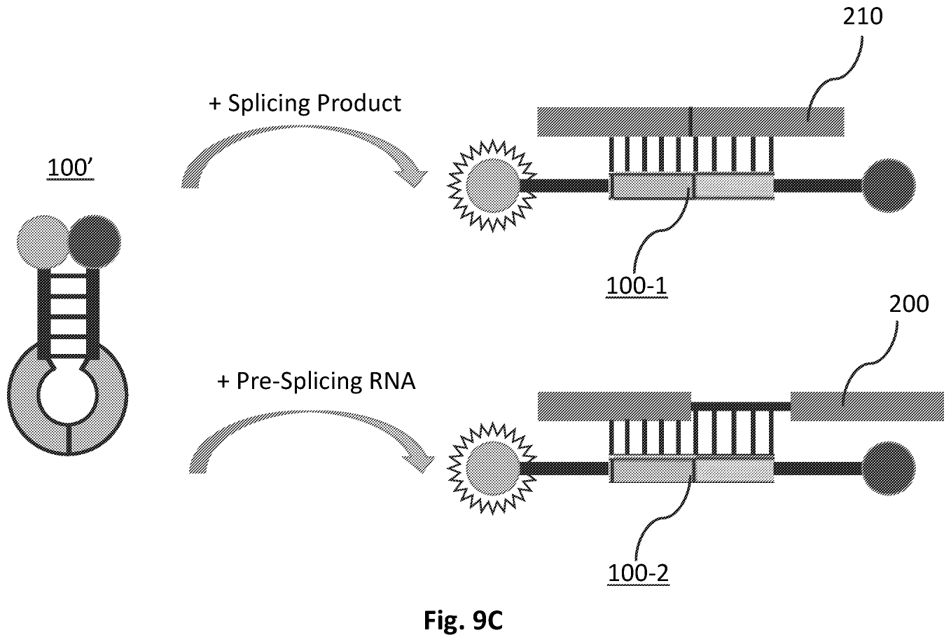

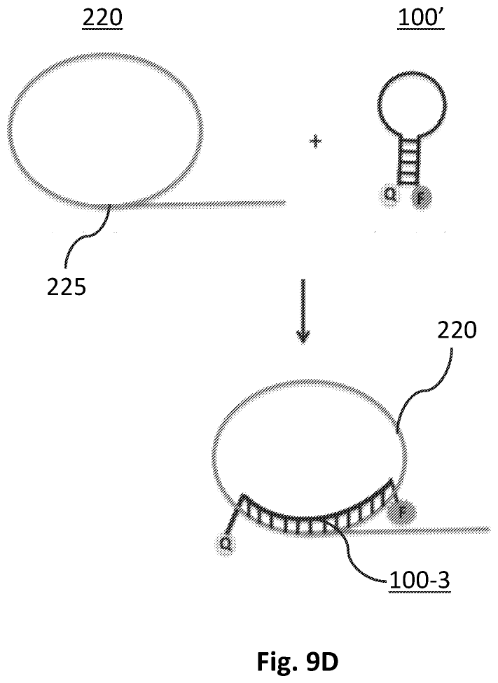

Referring to FIG. 9A, in some embodiments, the probe 100 includes a nucleic acid 110, a first fluorescent element 131 attached to a first end of the nucleic acid 110, and a second fluorescent element attached to a second end of the nucleic acid 110.

In some embodiments, the nucleic acid 110 comprises: a first stem portion 113; a second stem portion 115; and a loop portion 111 between the first stem portion 113 and the second stem portion 115.

In some embodiments, when the loop portion 111 is not hybridized (such as to a pre-splicing RNA molecule or a splicing product), the probe 100 forms a stem-loop structure 100′.

In some embodiments, in the stem-loop structure 100′, the first stem portion 113 and the second stem portion 115 are at least practically complementary to each other and form a stem structure 125. In some embodiments, when the loop portion 111 is not hybridized to the pre-splicing RNA molecule or the splicing product, the loop portion 111 forms a loop structure 115.

In some embodiments, in the stem-loop structure 100′, the first fluorescent element 131 and the second fluorescent element 133 are in proximity to each other (located in the same region 135), such that a fluorescent property of the first fluorescent element 131 is affected by the second fluorescent element 133.

Referring to FIG. 9B, a pre-splicing RNA molecule 200 includes exons 211 and 213, as well as intron 221 between the two exons. The pre-splicing RNA 200 undergoes splicing to produce a splicing product 210 (which includes the two exons but not the intron), as well as an intron product 220 (such as lariat intronic RNA).

Referring to FIG. 9C, in some embodiments, the probe 100 is a splicing product 210 specific probe 100-1. In the probe 100-1, the loop portion 111 of the nucleic acid 110 does not hybridize to the pre-splicing RNA molecule 200, and the loop portion 111 of the nucleic acid 110 hybridizes to the splicing product 210.

In some embodiments, the probe 100 is a pre-splicing RNA 200 specific probe 100-2. In the probe 100-2, the loop portion 111 of the nucleic acid 110 hybridizes to the pre-splicing RNA molecule 200, and the loop portion 111 of the nucleic acid 110 does not hybridize to the splicing product of the RNA molecule 210.

The splicing events involving any pre-splicing RNA molecule 200 and/or splicing product 200 can be studied using the probe 100; the instant specification does not intend to limit the scope of the splicing events. Alternative splicing and/or splicing defects presents in the development and tissue differentiation of organisms (Baralle et al., Nature Reviews Molecular Cell Biology 18, pages 437-451 (2017)). Misplicing of RNA is implicated in various diseases (see e.g., Scotti et al, Nature Reviews Genetics 17, pages 19-32 (2016)) such as cancer (see e.g., Bonnal et al., Nature Reviews Clinical Oncology 17, pages 457-474 (2020)), neurodegenerative diseases (see e.g., Li et al., Translational Neurodegeneration 10, Article number: 16 (2021)), cardiac disorders (Beqqali, Biophys Rev. 2018 August; 10(4): 1061-1071), autoimmune diseases (Ren et al., Front. Immunol., 17 Aug. 2021) and others. In many cases, the defects in RNA splicing are the causes or contributing factors of the diseases or disorders. Furthermore, splicing occurs in most eukaryotic genes (and some prokaryotic genes). As such the probe 100 can be designed according to the teachings of the instant specification to monitor individual splicing events in each scenario in all types of organisms. Because the probe 100 is able to, among others, detect and quantify the pre-splicing RNA molecule 200 and/or splicing product 200, the nature of the splicing (e.g., spliceosome splicing in mammals or autocatalytic RNA ribozymes in bacteria, fungi, plants, and protists) does not affect the effectiveness of the probe 100.

Any compounds or chemical structures that are able to hybridize to the splice site of interest is considered within the scope of the nucleic acid 110. In some embodiments, in the probe 100, the nucleic acid 110 includes RNA. In some embodiments, the nucleic acid 110 includes DNA. In come embodiments, the nucleic acid includes a modified nucleic acid. Examples of modified nucleic acids include a locked nucleic acid (LNA), a peptide nucleic acid (PNA), and the like. Modified nucleic acids are sometimes included in the nucleic acid 110 due to the stability, binding affinity, and/or other characteristics of the modified nucleic acids. Based on the teachings of the instant specification and the requirements of the specific applications, one of ordinary skill in the art would understand whether to choose modified nucleic acids over the natural nucleic acids, or which ones to choose. Modified nucleic acids are described in, in a non-limiting example, Nakatani et al. (Modified Nucleic Acids, Springer International Publishing, January 2016) Duffy et al. (BMC Biology 18, Article number: 112 (2020), the entirety of the reference is hereby incorporated herein by reference).

In some embodiments, the first fluorescent element 131 is a fluorophore and the second fluorescent element 133 is a quencher that absorbs a fluorescence emission from the fluorophore. According to these embodiments, when the probe 100′ is not in contact with the hybridization target, the fluorophore and the quencher are in proximity and located in the same region 135 due to the formation of the stem 125 by the first stem portion 113 and the second stem portion 115. Due to the limited distance between the fluorophore and the quencher, the quencher quenches the fluorescence signal of the fluorophore and the probe 110′ gives no fluorescence signal or weak fluorescence signal when irradiated by an excitation light. When the probe 100 is hybridized with an RNA molecule (either a pre-splicing RNA molecule or a splicing product), the hybridization between the loop portion 111 and the target breaks the stem 125 formed by the first stem portion 113 and the second portion 115 and separates the fluorophore from the quencher. Due to the much greater distance between the fluorophore and the quencher, the fluorophore is freed (at least partially) from the quenching and the probe 100 is able to give stronger fluorescence signal.

In some embodiments, the first fluorescent element is a donor in a fluorescence resonance energy transfer (FRET) pair, and the second fluorescent element is an acceptor in the FRET pair. According to these embodiments, when the probe 100′ is not in contact with the hybridization target, the donor and the acceptor are in proximity and located in the same region 135 due to the formation of the stem 125 by the first stem portion 113 and the second stem portion 115. Due to the short distance between the donor and the acceptor, the efficiency of the energy transfer is high and the acceptor is able to generate strong emission when the donor is shined with an excitation light. When the probe 100 is hybridized with an RNA molecule (either a pre-splicing RNA molecule or a splicing product), the hybridization between the loop portion 111 and the target breaks the stem 125 formed by the first stem portion 113 and the second portion 115 and separates the donor from the acceptor. Due to the much greater distance between the donor and the acceptor, the energy transfer efficiency is low and the acceptor is not able to generate emission or only weak emission when the donor is shined with the excitation light.

Referring to FIG. 9C, in some embodiments, the loop portion 111 of the probe 100-1 hybridizes to a segment of the splicing product 210 of the RNA molecule, and the segment of the splicing product spans at least two consecutive exons. In some embodiments, the loop portion 111 of the probe 100-2 hybridizes to a segment of the pre-splicing RNA molecule 200, and the segment of the pre-splicing RNA molecule spans at least one exon and at least one intron adjacent to the at least one exon. Referring to FIG. 9D, in some embodiments, instead of hybridizing to the splicing product 210 including the exon(s), the loop portion 111 of the probe 100-3 hybridizes to the lariat intronic RNA 220. In some embodiments, the loop portion 111 of the probe 100-3 hybridizes to the lariat intronic RNA 220 in a manner that the point 225 where the 5′-end and the branchpoint adenosine residue of the intron 221 join during the splicing process is within the double stranded structure formed by the hybridization.

In some embodiments, in the probe 100, a length of the first stem portion 113 or a length of the second stem portion 115 ranges from about 3 nucleotides to about 15 nucleotides, about 3 nucleotides to about 10 nucleotides, about 4 nucleotides to about 8 nucleotides, or about 3, 4, 5, 6, 7, 8, 9, 10, 11, 12, 13, 14, or 15 nucleotides.

In some embodiments, in the loop portion 111, a number of nucleotides complementary to the pre-splicing RNA molecule or the splicing product ranges from about 10 to about 30, such as about 12 to about 20, or about 10, about 12, about 14, about 16, about 18, about 20, about 22, about 24, about 26, about 28, or about 30.

In some embodiments, a melting temperature of a hybridization product formed by the probe 110 and the pre-splicing RNA molecule 200 or the splicing product 210 ranges from about 40° C. to about 85° C., such as about 45° C. to about 70° C., about 50° C. to about 65° C., about 60° C. to about 75° C. Melting temperatures are affected by extrinsic factors other than the nucleotide sequences. Therefore, the melting temperatures as described herein are measured or predicted according to the conditions described in the “Definition” section.

In some embodiments, in the probe 100, the nucleic acid 110 further comprises a linker. In some embodiments, the linker is at the first end of the nucleic acid, at the second end of the nucleic acid, between the first stem portion 113 and the loop portion 111, and/or between the loop portion 111 and the second stem portion 115. In some embodiments, a length of the linker ranges from about 1 nucleotide to about 15 nucleotides, such as about 1, about 2, about 3, about 4, about 5, about 6, about 7, about 8, about 9, about 10, about 11, about 12, about 13, about 14, or about 15 nucleotides

In some embodiments, the probe 110 does not include any nucleotides except for nucleotides in the nucleic acid 110. In some embodiments, the nucleic acid 110 includes only nucleotides. In some embodiments, the nucleic acid 110 includes only the loop portion 111, the first stem portion 113 and the second stem portion 115. In some embodiments, all nucleotides in the loop portion 111 are complementary to the pre-splicing RNA molecule or the splicing product. In some embodiments, the first stem portion 113 and the second stem portion 115 are fully complementary to each other.

### Method of Monitoring RNA Splicing

The present study developed probes for monitoring specific RNA splicing events, and further demonstrated that the probe is able to monitor both the specificity and the splicing kinetics and suitable for used in high throughput screening for agonists and antagonists of splicing.

Accordingly, in some aspects, the instant invention is directed to a method of monitoring RNA splicing.

In some embodiments, the method includes preparing a mixture including a pre-splicing RNA molecule and a probe for monitoring RNA splicing. In some embodiments, a change of a fluorescence signal of the probe indicates a splicing of the RNA molecule. In some embodiments, the probe, and/or the manner by which the probe works, is the same as or similar to those as detailed in the “Probe for Monitoring RNA Splicing” section.

In some embodiments, the mixture is an in vitro mixture outside a cell and/or a subject. In some embodiments, the mixture is inside a cell or inside a subject. One of ordinary skill in the art would understand that the probe can be introduced into a cell, such as a cell in a subject or an isolated cell, by various means including microinjection, liposome, nanoparticles, and the like, thereby mixing with RNA molecules in the cell.

In some embodiments, a change of a fluorescence signal of the probe indicates a splicing of the RNA molecule. In some embodiments, an increased level of a fluorescence signal indicates that the splicing has taken place. In some embodiments, a decreased level of the fluorescence signal indicates that the splicing has taken place.

In some embodiments, an amount of a change in the fluorescence signal is proportional to a degree of the splicing of the RNA molecule.

In some embodiments, a level of the fluorescence signal of the probe corresponds to a level of the pre-splicing RNA molecule or a level of a splicing product of the pre-splicing RNA molecule.

As detailed above, alternative splicing is found in, among others, development and tissue differentiation of organisms. Defects in the splicing processes are implicated in various types of diseases and disorders, such as cancers, neurodegenerative diseases, cardiac disorders, an autoimmune disease, and the like. Furthermore, certain RNA splicing events in some pathogenic organisms are required for the survival of the pathogenic organisms.

Accordingly, in some embodiments, the method of monitoring RNA splicing is a method for diagnosing a disease or a disorder, or monitoring a state of a disease or a disorder, wherein the disease or disorder is caused by or involves a RNA splicing event not present in a healthy cell, a healthy tissue, or a healthy subject, and wherein the probe detects the RNA splicing event. In some embodiments, the disease or disorder is a cancer, a neurodegenerative disease, a cardiovascular disorder, an infection by a pathogen, or the like.

In some embodiments, the method of monitoring RNA splicing is a method for studying a developmental process or a tissue differentiation process or state, wherein the developmental process or the tissue differentiation process or state involves a RNA splicing event specific to the developmental process, the tissue differentiation process or the tissue differentiation state.

In some embodiments, the method of monitoring RNA splicing further includes applying a DNA/RNA editing technology to the system in which the RNA splicing is being monitored. Detailed of the combination of the probe and the DNA/RNA editing technology is described in the “Combination with DNA/RNA Editing Technology” section herein.

### Method of Screening Modulators of RNA Splicing

Many defects in RNA splicing are the causes or contributing factors of various diseases and disorders, such as neurodegenerative diseases, development disorders, cardiac disorders, autoimmune diseases, cancers, and the like. Modulators of RNA splicing have been tested as therapies for neurodegenerative diseases (Li et al., Translational Neurodegeneration 10, Article number: 16 (2021)), anti-cancer agents (Effenberger et al., Wires RNA, Volume 8, Issue 2 March/April 2017 e1381). Furthermore, RNA splicing is present in virtually all eukaryotic pathogens. Disrupting RNA splicing events are important to the eukaryotic pathogens can potentially disrupt the function of the pathogenic organism and cause the death or incapacitation of the organism. For example, Group I and Group II introns are found in the genome of many pathogenic fungal organisms, and small molecule modulators of their splicing could become a promising new class of antifungal drugs.

The present study designed probes for monitoring RNA splicing, and further demonstrated that the probe is able to monitor the splicing kinetics and suitable for used in high throughput screenings.

Accordingly, in some aspects, the instant invention is directed to a method of screening modulators of RNA splicing.

In some embodiments, the method includes preparing a first mixture including a pre-splicing RNA molecule, and a probe for monitoring the formation of a specific set of spliced exons; determining a first fluorescence signal level change of the first mixture; preparing a test mixture including the pre-splicing RNA molecule, the probe of claim 1 and a test compound; and determining a second fluorescence signal level change of the second mixture. In some embodiments, a difference between the first fluorescence signal level change and the second fluorescence signal level change indicates that the test compound is a modulator of the splicing of the RNA molecule.

In some embodiments, an increased level of a fluorescence signal indicates that splicing of specific exon sequences has taken place. In some embodiments, a decreased level of the fluorescence signal indicates that the specific splicing event has taken place. In some embodiments, an amount of a change in the fluorescence signal is proportional to a degree of the splicing of the RNA molecule.

In some embodiments, the test compound is a modulator of a given RNA splicing event if splicing kinetics of the RNA changes in the presence of the test compound. In some embodiments, the test compound is an inhibitor of a specific RNA splicing event if a rate or extent of the splicing of a given spliced product decreases. In some embodiments, the test compound is an enhancer of a specific RNA splicing event if the rate or extent of the splicing of the RNA molecules increases.

In some embodiments, the splicing event is a splicing event specific to a developmental process or a differentiated tissue or cell.

In some embodiments, the splicing event is a splicing event specific to a disease or a disorder.

In some embodiments, the splicing event occurs specifically during cancer. RNA splicing specific to various types of cancer is known in the art and is described in, e.g., Zhang et al. (Signal Transduction and Targeted Therapy volume 6, Article number: 78 (2021)) and Marabti et al. (Front. Mol. Biosci., 7 Sep. 2018).

In some embodiments, a given splicing event is specific to a non-mammalian organism. In some embodiments, the splicing of the RNA molecule is specific to a non-human organism. In some embodiments, the splicing of the RNA molecule is specific to an organism pathogenic to a mammal, or a human. In some embodiments, the splicing of the RNA molecule is specific to a fungal organism, such as a pathogenic fungal organism, such as a fungal organism pathogenic to a mammal, such as a human.

### Combination With DNA/RNA Editing Technology

In some embodiments, the probe as described herein is used in conjunction with a DNA/RNA editing technology. In some embodiments, the editing technology is one that affects an amount and/or a sequence of a pre-splicing RNA molecule or a splicing product, such as a pre-splicing RNA molecule or a splicing product that interact with the instant probe and changes the fluorescence characteristics of the probe. In some embodiments, the probe provides a readout for the editing technology. In some embodiments, the probe provides a readout for the editing technology in a cell.

In some embodiments, the probe as described herein is used as a readout for the editing technology, such as a readout for the efficiency of the editing. In some embodiments, the probe as described herein is used as a readout for efficiency of the DNA/RNA editing at the same time of monitoring the RNA splicing.

In some embodiments, the DNA/RNA editing technology includes an RNA interference technology, a CRISPR based editing technology, or the like. Examples of RNA interference technology includes siRNA, shRNA, miRNA, and the like. Examples of CRISPR based editing technology includes CRISPR-Cas9, CRISPR-Cas12, CRISPR-Cas13, CRISPR-Cas3, and the like.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

Although the non-limiting exemplary probe as well as methods thereof described in the “Examples” section herein are designed to monitor an autocatalytic group II intron found in the mitochondrial large subunit ribosomal ribonucleic acid (LSU rRNA) of Histoplasma capsulatum (H. capsulatum), it would be apparent to one of ordinary skill in the art that the probe and methods described herein are applicable to all types of splicing events, including spliceosome RNA splicing commonly found in mammals such as humans, as well as other types of autocatalytic intron splicing.

### Example 1: Group II Introns, Human Pathogens and Molecular Beacons

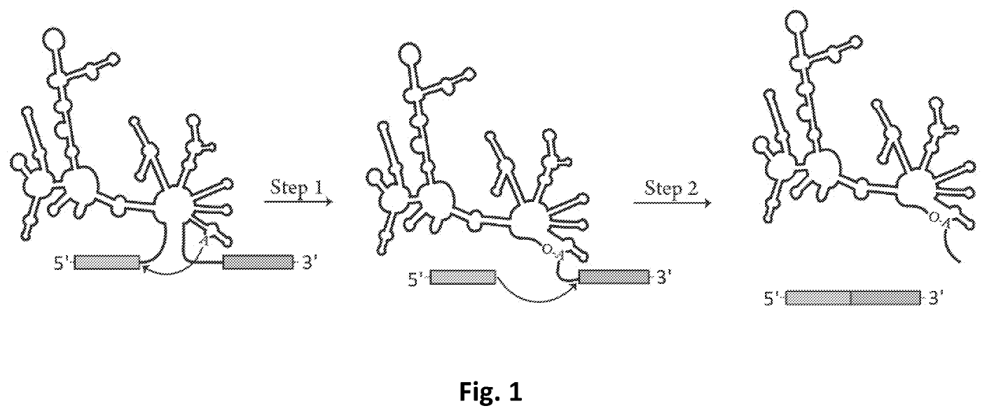

Group II introns are a class of autocatalytic ribozymes found in bacteria and the organellar genomes of fungi, plants, and protists. These introns represent an ancient predecessor of the eukaryotic spliceosome, which shares the same characteristic two-step sequential splicing mechanism (FIG. 1). Despite a lack of phylogenetic conservation at the sequence level, group II introns have highly conserved secondary and tertiary structures. Because of their distinctive structure and ubiquity in mitochondrial genes responsible for the respiration of fungi, group II introns represent a promising therapeutic target that has largely been neglected by traditional screening and drug discovery platforms.

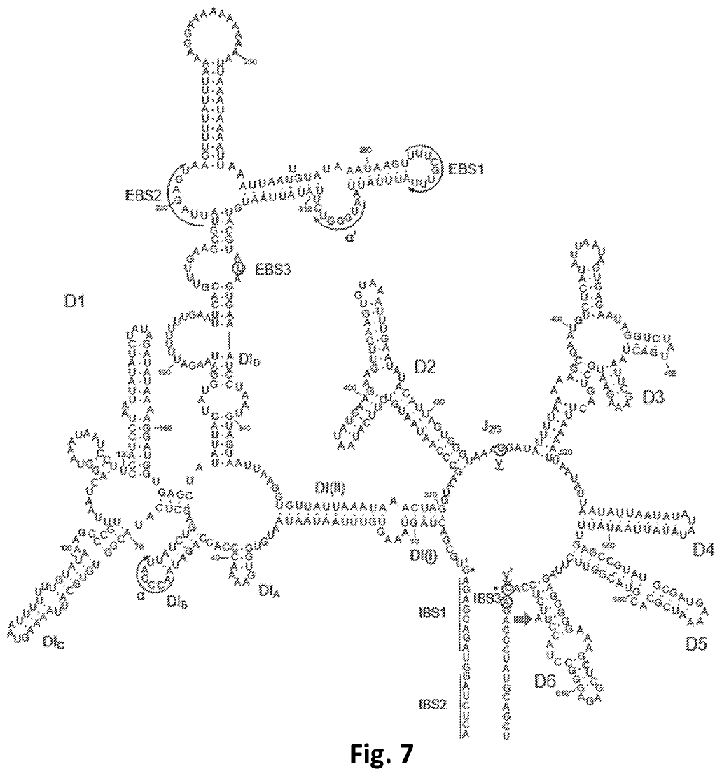

While small molecule targeting of RNA is a relatively new field, promising intron drug targets are being continually identified in human pathogens. One such intron, found in the mitochondrial LSU rRNA of Histoplasma capsulatum (H. capsulatum) (FIG. 7), was identified and shown to exhibit self-splicing activity in the presence of catalytic Mg²⁺ in vitro. Endemic to river valleys, H. capsulatum is a dimorphic fungus responsible for histoplasmosis, which is the most prevalent dimorphic fungal infection in the United States and a significantly underdiagnosed disease globally. The identified H. capsulatum group II intron from the mitochondrial ribosomal large subunit RNA (H.c.LSU) exhibits a relatively slower splicing rate constant relative to other group II introns.

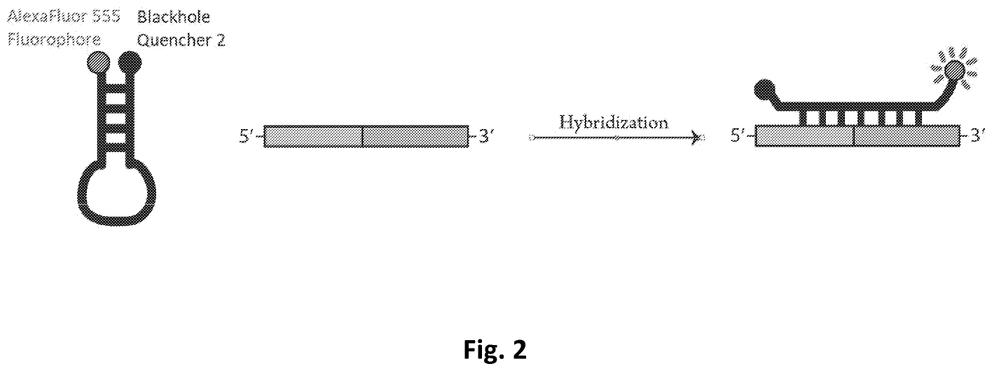

Molecular beacons are fluorescent DNA-based probes that can detect specific sequences by complementary hybridization. To achieve the most sensitive detection of nucleic acid sequences, molecular beacons are often designed as stemloop structures in which the fluorophore and quencher dyes are covalently attached at the termini of an oligonucleotide, in forced proximity with each other. Upon binding a complementary target sequence, however, the beacon will open and hybridize to the target, thereby increasing the distance between the fluorophore and the quencher and allowing fluorescence to serve as a signal for target identification in solution (FIG. 2). Molecular beacons have become a powerful chemical biology tool, and they are commonly used to study RNA cellular localization and intronic structure. At least one high-throughput screening assay based on molecular beacons has been developed to identify modulators of RNA targets, such as miRNAs.

Here it is demonstrated that molecular beacons can provide a reliable readout of RNA splicing kinetics and that the resulting methods are sufficiently generalizable and sensitive for application in high-throughput assays for identification of small molecule splicing inhibitors. Implementation of this assay for monitoring splicing and inhibition of the H.c.LSU group II intron provides guidance to the creation of improved assays for small molecule targeting of self-splicing and pre-mRNA introns.

### Example 2: Materials and Methods

### Molecular Beacon and Target Synthesis

RNA oligonucleotide HCSE (5′-ACUCUAGGUAGACGAGAAGACCCUAUGCAGCU-3′, SEQ ID No: 1) was synthesized on a MerMade 12 DNA-RNA synthesizer (BioAutomation) using TBDMS RNA phosphoramidites (TxBio), deprotected and purified on an 18% denaturing polyacrylamide gel as previously described (Wincott et al., Nucleic Acids Research, 23, 2677-2684 (1995) and Dickey et al, Nucleic Acids Research, 45, 11980-11988 (2017)).

DNA oligonucleotides MB14 (5′-Am-CCAGGAGGGTCTTCTCGTCCCTGG-BHQ2-3′, SEQ ID No.: 2), MB18 (5′-Am-CCAGGATAGGGTCTTCTCGTCTACCTGG-BHQ2-3′,SEQ ID No.: 3) containing the 3′-terminal Black Hole Quencher 2 (BHQ2) and 5′-terminal aminomodifier C3 TFA (Am) (Glen Research) were synthesized on a MerMade 12 DNA-RNA synthesizer (BioAutomation) using UltraMild Base protection DNA phosphoramidites (Glen Research). The oligonucleotides were then deprotected using 28-30% ammonium hydroxide (J.T. Baker) at room temperature for 24 h and purified on an 18% denaturing polyacrylamide gel.

### Fluorescent Labeling of Molecular Beacons

Purified MB14 and MB18 containing BHQ2 at the 3′-end and the aminomodifier C6dT nucleotide at the 5′-end were covalently attached to the NHS ester of the AlexaFluor 555 dye (Life Technologies Corp.) via the primary amino group on the aminomodifier nucleotide. The beacons were dissolved in a 200 μL solution of 0.25 NaHCO₃ buffer (pH 9.2) before being combined with a 200 μL formamide solution containing 0.5 mg of the AlexaFluor 555 NHS ester dye. The labeling reaction was allowed to proceed at room temperature for 2 h, and the fluorescently labeled products were then purified on a 18% denaturing polyacrylamide gel and stored in RNA storage buffer.

### Detection of the Spliced Exons Using Molecular Beacons

Wells of black 96-well plates (Corning 3792) were filled with 50 μL of solution containing 100 nM HCSE or 200 nM H.c.LSU, 50 mM HEPES (pH 7.5), 150 mM NH₄Cl, and 10 mM MgCl₂ in water (optimal reaction conditions). Where indicated, 10 mM MgCl₂ was omitted or 10 mM EDTA was added to the reaction mixture. The reaction mixture was incubated at 37° C. for 2 hours. Then, 50 nM of MB14 or MB18 was added to each well. The plate was then heated to 70° C. for 5 min and incubated at 37° C. for 30 min before analysis on a Synergy H1 plate reader (BioTek).

### Determination of Self-splicing Reaction Rate Constants

For each reaction, 10 wells of black 96-well plates (Corning 3792) (corresponding to 10 time points) were filled with 50 μL of a solution containing 200 nM HC preRNA (unlabeled or 32P-body labeled), 50 mM HEPES (pH 7.5), 150 mM NH₄Cl, and 10 mM MgCl₂ in water. The reaction was allowed to run at 37° C. At each time point, 10 mM EDTA was added to the respective well in order to quench the splicing reaction. Once the time course was completed and all reaction wells were quenched, 50 nM MB18 was added to each well. The plate was then heated to 70° C. for 5 min and allowed to cool to 37° C. for 30 min before analysis on a Synergy H1 plate reader (BioTek). The GraphPad Prism software package was used to fit the self-splicing time course data to an exponential function that accounts for a time lag: A*e (−B(t−C))+D, where A represents the y-intercept, B was extracted as the reaction rate constant kobs, C was the time lag parameter, and D represents a vertical shift parameter.

In order to compare the reaction rate constants determined using the radioanalytical and molecular beacon assays, 32-P body-labeled precursor RNA was added to the reaction, and 1 μL was withdrawn from each aliquot prior to MB18 addition for analysis and visualization on a 5% denaturing polyacrylamide gel. Bands corresponding to the precursor and splicing products were visualized on an Amersham Typhoon phosphorimager and quantified using the ImageQuant TL imaging software package. The fraction of spliced exons was then plotted over time and fit to a simple exponential equation for reaction rate constant determination as described.

### Quantitative Analysis of Self-Splicing Reaction on Sequencing Gels.

1 μL aliquots of each quenched self-splicing time point were mixed with 4 μL dye before loading onto a 5% denaturing polyacrylamide gel. After running the gel at 25 W for 90 min, the gel was then dried and exposed to a phosphor screen overnight before analysis on an Amersham Typhoon phosphorimager. Radioactivity of individual bands was quantified using the ImageQuant TL imaging software package, and the signal in each band was corrected by normalizing with respect to the number of radioactive uridine nucleotides present in each splicing product. The fraction of spliced exons was then plotted over time and fit to a simple exponential for reaction rate determination as described.

### K_{I} Measurement for Mitoxantrone Inhibition of the Self-Splicing Reaction.

Reaction rate constants were determined in the presence of 14 different mitoxantrone concentrations, ranging from 5 nM to 300 μM, using MB18 as described above. The derived reaction rate constants were plotted against the respective inhibitor concentrations, and the GraphPad Prism software package was used to fit the data to a four-parameter logistic function A+(B−A)/(1+(x/C)D), where A is the minimum response, B is the maximum response, C is the KI, and D is the slope parameter.

### Example 3: Selected Results

### Example 3-1: Design of Molecular Beacons Targeting Spliced Exons

Based on the principles of molecular beacon design (see e.g., Tsourkas et al., Nucleic Acids Research 2002, 30 (19), 4208-4215 and Tsourkas et al., Nucleic Acids Research 2003, 31(4), 1319-1330, the entireties of the references are incorporated herein by reference) and understanding of the H.c.LSU group II intron, two DNA beacons designed to hybridize specifically to the junction formed when the 5′-and 3′-exons are ligated together in the second step of the sequential self-splicing reaction were synthesized (FIGS. 1-2). The stem length of both beacons was kept constant at five nucleotides because this length ensured low background fluorescence without compromising the rate of hybridization with the target. The length of the loop portion complementary to the ligated spliced exons was varied; however, in order to test the activity and specificity dependence of the designed beacons. While beacons with longer loop lengths tend to have more stabilized interactions with their target sequence, this greater length also risks decreasing their binding specificity, as longer beacons are more likely to hybridize with off-target sequences that share some identity with the target. Taking these considerations into account, the shorter beacon was designed with a loop length of 14 nucleotides (MB14), while the longer beacon had a loop length of 18 nucleotides (MB18). To aid in optimizing the beacon assay and to serve as a positive control for beacon hybridization to the spliced exons, a synthetic 32-nucleotide RNA oligo (HCSE) with the same sequence identity as the spliced exons junction was also prepared.

### Example 3-2: Molecular Beacons Selectively Recognize Ligated Exon Products

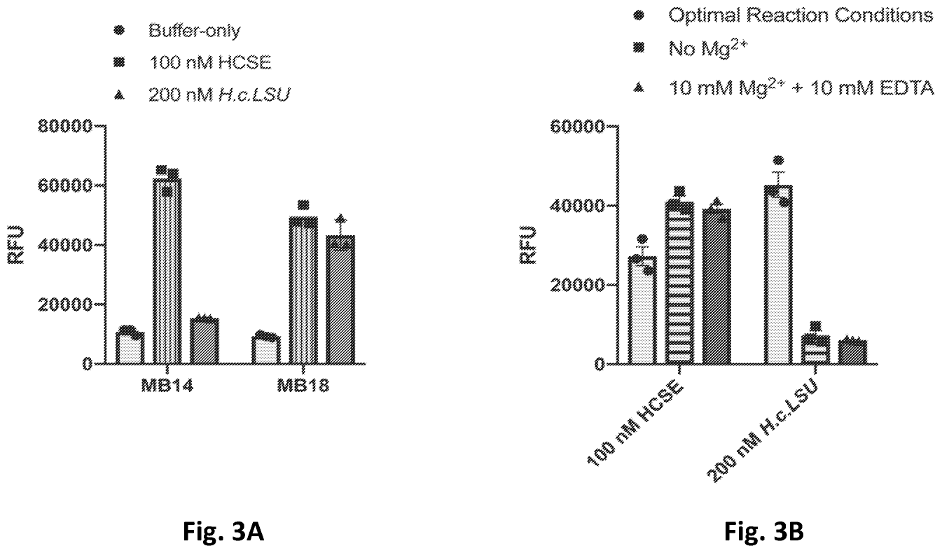

To confirm that the designed beacons behave as expected in the presence of the target sequence (spliced exons), the activities of MB14 and MB18 at 50 nM were assayed in the presence of 100 nM HCSE target oligonucleotide (FIG. 3A), which has the same sequence as spliced exons. Notably, both beacons exhibited high fluorescent signal upon binding the HCSE target sequence.

Whether the beacons could detect the specific sequences of individual ligated exons

formed during the course of the corresponding intron self-splicing reaction was then tested. The reaction was carried out under the optimal conditions for splicing via branching (see “Example 2” section herein).

The HC preRNA was studied at 200 nM to ensure sufficient accumulation of ligated exons such that at least a five-fold signal to noise ratio upon hybridization with the beacon would be expected. After the splicing reaction was carried out for two hours, 50 nM of beacon was added and the reaction mixture was denatured at 70° C. for 5 min. Then the samples were incubated at 37° C. for 30 min to allow the beacon to hybridize to the target. It was found that, under these conditions, MB18 produced strong fluorescent signal upon binding the ligated exons that are generated during the splicing reaction, but the signal for MB14 was weaker (FIG. 3A). Based on these results, MB18 was chosen for further experiments.

Since MB18 can potentially hybridize to the precursor RNA by binding to the last 9 nucleotides of the 5′-exon and then reaching across the intron to hybridize with first 9 nucleotides of the 3′-exon, it was important to test its ability to differentiate between the precursor RNA and the ligated exons. For this purpose, a variation of the experiment described above in which the mixture contained no Mg²⁺ ions was carried out, thereby precluding self-splicing of precursor RNA. However, the reaction contained monovalent ions (150 mM NH₄Cl), which are sufficient for promoting RNA and DNA duplex formation. In the absence of Mg²⁺, solutions containing the HC preRNA and MB18 displayed only background fluorescence, at a level comparable to that of samples that lacked spliced exon sequences (FIG. 3B, right). At the same time, the absence of Mg²⁺ ions did not affect the ability of the MB18 to hybridize with the synthetic RNA target oligonucleotide identical to the spliced exons (FIG. 3B, left). These data indicate that MB18 selectively binds to the ligated exons and not to the precursor RNA.

EDTA is commonly used to quench splicing reactions because it sequesters the magnesium ions required for splicing catalysis. When 10 mM EDTA is added together with 10 mM Mg²⁺ prior to incubation, a solution containing MB18 beacon and the HC preRNA displays only background fluorescence, as if Mg²⁺ were not present in the solution (FIG. 3B, right). This control demonstrates that EDTA does not impede hybridization of the beacon to the target, evidenced by similar levels of activity towards the HCSE oligo in wells with or without the sequestering agent (FIG. 3B, left), but it does quench the splicing reaction. These data indicate that equimolar amounts of EDTA relative to Mg²⁺ provide an effective quench of the splicing reaction.

### Example 3-3: Application of Molecular Beacons for Monitoring Splicing Kinetic

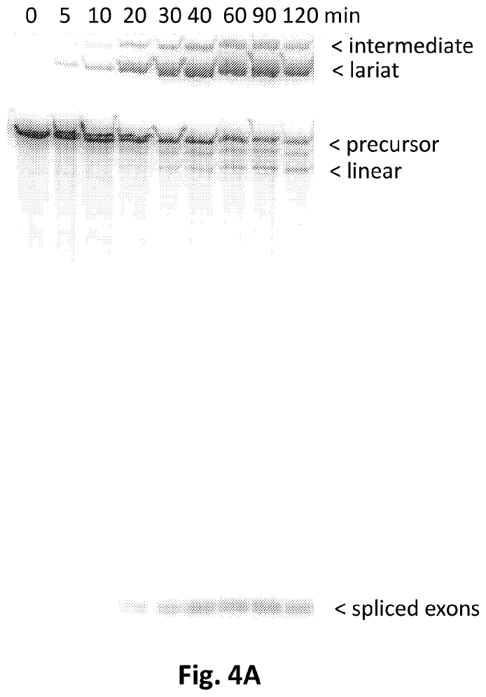

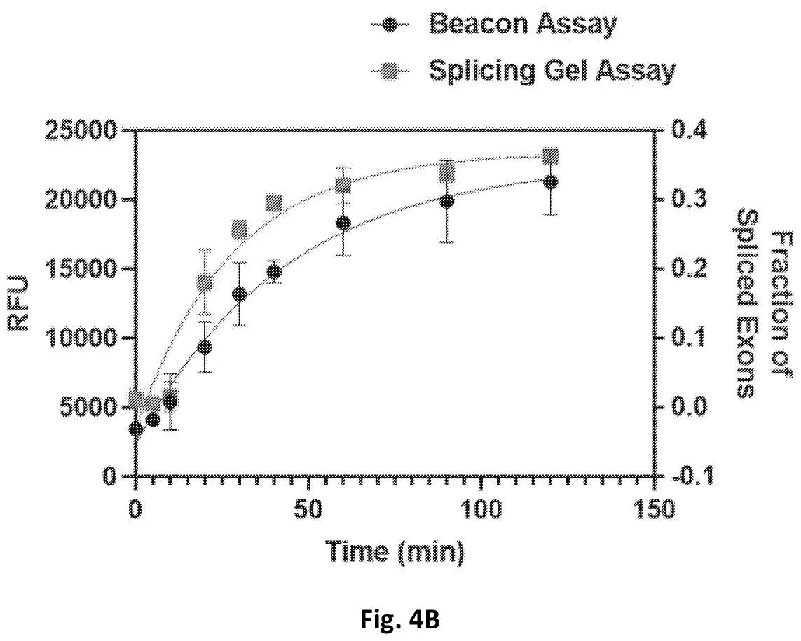

Kinetic characterization of self-splicing is typically performed using radioanalytical methods, in which the precursor RNA is internally labelled with a ³²P-α-NTP and progress of the splicing reaction is monitored over time by electrophoretic separation of reaction products. In order to determine if accumulation of spliced exons can be accurately measured using molecular beacons, the same splicing reaction was monitored using both the radioanalytical and molecular beacon fluorescent methods. In this experiment, ten reaction chambers (plate wells) were filled with a solution of 200 nM of cold HC preRNA, which had been spiked with 1 nM of [α-³²P]-UTP body-labeled HC preRNA, and the splicing reaction was initiated by adding a solution of MgCl₂ (10 mM final concentration). Subsequently, the reaction in each well was quenched at specified time points by addition of 10 mM EDTA. The contents of each well were then split and analyzed with the two analytic methods in parallel. For the radioanalytical splicing assay, 5 μl of solution from a given well was mixed with dye, and the products were separated and visualized on a 5% denaturing polyacrylamide gel (FIGS. 4A-4B). For the MB18 assay, the remaining contents of each well (45 μl) were combined with 5 μl of a MB18 beacon solution (50 nM final concentration), beacon was allowed to anneal, and the results were analyzed on a plate reader, as described in “Example 2” section (FIG. 4B). When data from the two methods were compared, changes in MB18 fluorescent signal that correlated directly with the accumulation of radiolabeled spliced exons were observed (FIG. 4B), ultimately resulting in similar rate constants (0.036±0.009 min⁻¹ for radioanalytical and 0.023±0.009 min⁻¹ for the beacon assay). These data indicate that the molecular beacon assay can be used to accurately monitor the splicing of RNA precursors.

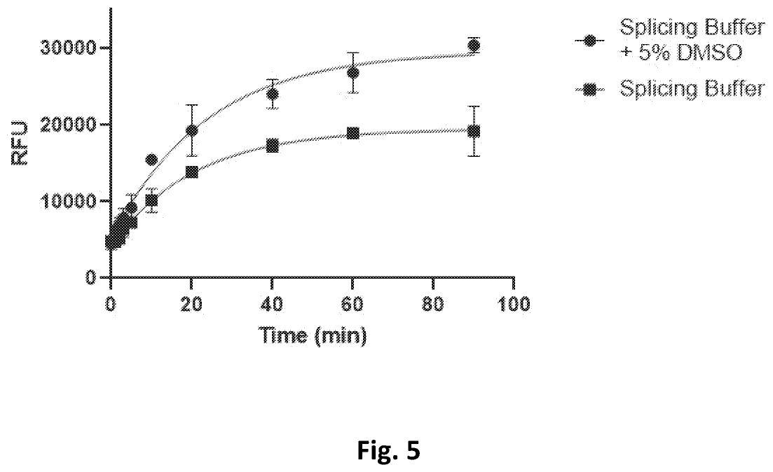

Given that the testing of small molecule inhibitors often involves dissolution of compounds in DMSO, it was important to test the impact of 5% dimethyl sulfoxide (DMSO) on the progression of the splicing reaction and the ability of MB18 to detect spliced exon product. It was observed that addition of 5% DMSO to the reaction had no deleterious effect on detection and actually increased the signal-to-background ratio of the beacon assay, resulting in a ˜6-fold window for a 90 min time course (FIG. 5). That 5% DMSO improves activity of the MB18 beacon is consistent with previous studies of molecular beacon fluorescence activity in the presence of organic solvents, where it has been shown that solvents like DMSO decrease the activation energy required for the strand hybridization reaction. It is notable that DMSO has no significant impact on the splicing reaction kinetics, as reaction rates of 0.043±0.013 min⁻¹ and 0.048±0.012 min⁻¹ in the presence and absence of DMSO were observed, respectively. These findings establish the utility of the beacon method for high-throughput screening of and kinetic characterization of small molecule splicing inhibitors.

### Example 3-4: Determination of a Small Molecule Inhibition Constant Using the Molecular Beacon Assay

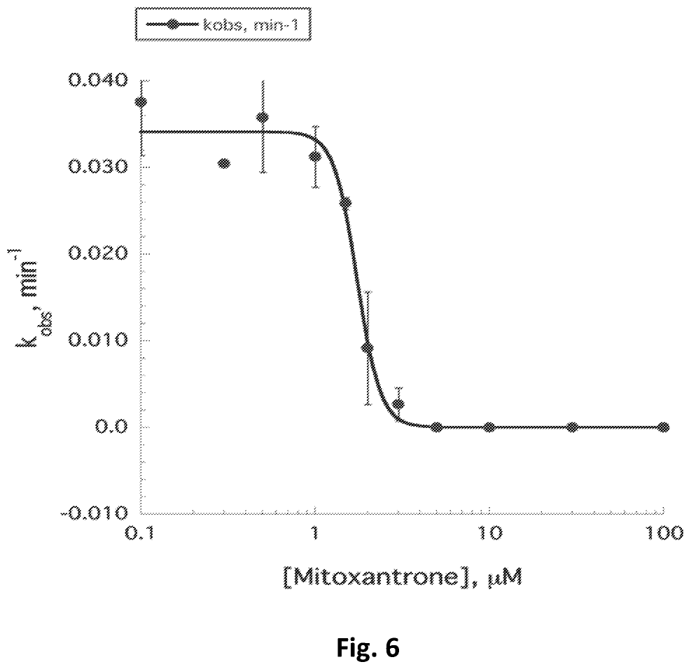

Given that the molecular beacon assay can be used to accurately determine splicing rate constants, the present study set out to determine if the assay can be used to measure small molecule inhibition of splicing under conditions amenable to high throughput analysis. Since there are no known inhibitors of the H.C. LSU group II introns, inhibition with the non-specific RNA binder mitoxantrone was tested (FIG. 6). Originally discovered as a strong binder of stem loop RNAs, mitoxantrone non-specifically inhibit splicing reactions by intercalating within RNA junction structures, making it a useful tool compound for K_{I} determination using our novel beacon assay.

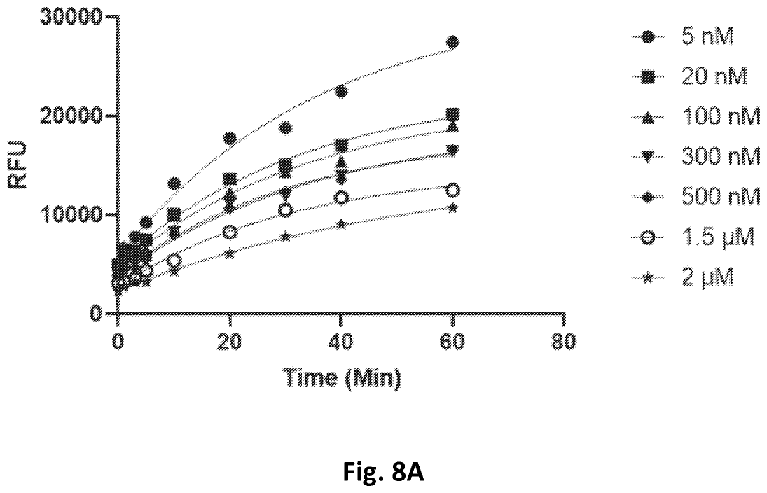

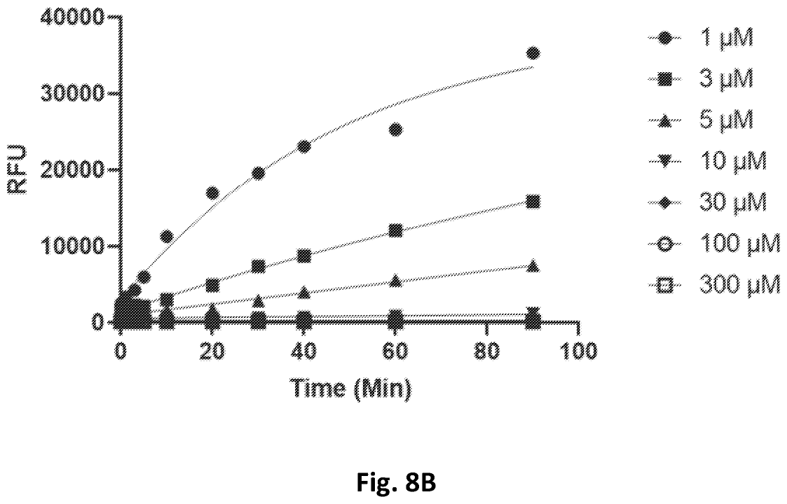

To test the effect of mitoxantrone, MB18 was used to monitor the efficiency of splicing in the presence of increasing drug concentrations. Reaction rate constants were plotted against the concentration of the inhibitor to yield an inhibition constant of 1.79±0.32 μM (FIGS. 6 and 8A-8B), indicating strong inhibition of the splicing reaction. This experiment demonstrates that the molecular beacon platform allows for construction of a typical dose-response curve, with characteristic plateaus and a sloped regions needed for quantitative analysis.

Though the Example section focuses on the design and application of molecular beacons that are sensitive to the second and final step of RNA splicing (exon ligation), although the assay can be easily adapted for use in other systems. For example, it is also possible to design beacons for monitoring formation of the lariat-3′-exon intermediates or 5′-exon-intron junctions, which would facilitate analysis of the first step of self-splicing. Though only one beacon is necessary for high-throughput screening purposes, synthesis of alternative beacons could enable independent determinations of individual reaction rates in a multiplexed assay.

### Example 4: Comments

The Example section describes, as a non-limiting example, the development of a novel molecular beacon assay for the study of group II self-splicing reactions. Using the H.c.LSU construct as a model, two beacons were synthesized with 14- and 18-nucleotide loop sequences complementary to the junction of the spliced exons product evolved in the course of the group II intron's splicing reaction. Assays of MB14 and MB18's activity towards the synthetic oligo target HCSE and the ligated spliced exons revealed that MB18 exhibits higher fluorescence activity without compromising specificity. By testing MB18 under various salt conditions and in the presence of the sequestering agent EDTA, it was shown that beacon activity towards the target sequence was not impeded by conditions required to initiate and quench the self-splicing reaction. Determination of reaction rate constants from time courses of the splicing reaction was achieved by interpreting fluorescence activity of MB18 as a proxy for the presence of spliced exons, a correlation confirmed by running a sequencing gel in parallel with beacon-based time courses to validate the latter assay. Additionally, it was found that 5% DMSO slightly improved beacon activity, an important step towards ensuring this assay's potential as a small molecule screening platform for group II self-splicing inhibitors. Finally, it was also shown that the beacon assay described herein can be used to independently determine K_{I} values for known inhibitors like mitoxantrone.

While it is important to test beacons that could alternatively hybridize to the precursor or lariat product of the splicing reaction in order to demonstrate the versatility of this approach, the results presented herein establishes this platform as a tool for small molecule discovery efforts towards not only the H.c.LSU construct but other splicing reactions as well.

Furthermore, while the beacon assay as described in the “Examples” section was optimized for monitoring self-splicing of group II introns, beacon assays for high-throughput screening and drug development can be readily designed for monitoring group I intron splicing (also common in fungal pathogens) and nuclear pre-mRNA splicing in metazoans (central to expression of specific genes). The design of beacons in other systems will require considerations of the particular characteristics of a target intron's structure, exon sequences and self-splicing reaction mechanism. For example, the S. cerevisiae ai5γ mitochondrial group II intron exhibits hydrolytic reopening of its spliced exons under certain reaction conditions. A molecular beacon designed for hybridization to the spliced exons of the ai5γ intron is expected to show relatively weak activity, and a probe that detects the formation of spliced lariat intron product could prove more effective. This is not a concern for group I or pre-mRNA introns, which do not generally undergo reopening of their spliced exons product but which by contrast might not form lariat splicing products. For such systems, recognition of spliced exons by molecular beacons would be most ideal.

The adaptability of the molecular beacon platform for monitoring various types of RNA splicing under high-throughput conditions will facilitate the screening of small molecules that target specific splicing reactions and isoforms.

### Enumerated Embodiments

In some aspects, the present invention is directed to the following non-limiting embodiments;

Embodiment 1: A probe for monitoring the sequence-specific splicing of individual RNA exons
the probe comprising a nucleic acid, a first fluorescent element attached to a first end of the nucleic acid, and a second fluorescent element attached to a second end of the nucleic acid,
wherein the nucleic acid comprises:
   a first stem portion;
   a second stem portion; and
   a loop portion,
   wherein one of the following applies:
      the loop portion of the nucleic acid does not hybridize to a pre-splicing RNA molecule and hybridizes to a splicing product of the RNA molecule, or
      the loop portion of the nucleic acid hybridizes to the pre-splicing RNA molecule and does not hybridize to the splicing product of the RNA molecule,
wherein the first stem portion and the second stem portion are at least practically complementary to each other and can form a stem structure when the loop portion is not hybridized to the pre-splicing RNA molecule or the splicing product, and
wherein the fluorescent signal of the probe when the loop portion of the nucleic acid hybridizes to the pre-splicing RNA molecule or the splicing product, and the fluorescent signal of the probe when the loop portion of the nucleic acid does not hybridize to the pre-splicing RNA molecule or the splicing product, are different.

Embodiment 2: The probe of Embodiments 1, wherein the nucleic acid is RNA, DNA, or a modified nucleic acid.

Embodiment 3: The probe of Embodiment 1, wherein, when the loop portion of the nucleic acid is not hybridized to the pre-splicing RNA molecule or the splicing product, the first fluorescent element and the second fluorescent element are in proximity to each other such that a fluorescent property of the first fluorescent element is affected by the second fluorescent element.

Embodiment 4: The probe of Embodiment 3, wherein at least one of the following applies:
the first fluorescent element is a fluorophore and the second fluorescent element is a quencher that decreases the fluorescence intensity of the fluorophore, or
the first fluorescent element is a donor in a fluorescence resonance energy transfer (FRET) pair, and the second fluorescent element is an acceptor in the FRET pair.

Embodiment 5: The probe of Embodiment 1, wherein at least one of the following applies:
the loop portion hybridizes to a segment of the splicing product of the RNA molecule, and the segment of the splicing product spans at least two consecutive exons,
the loop portion hybridizes to a segment of a lariat intronic RNA formed by the splicing, and the segment of the lariat intronic RNA comprises the point where the 5′-end and the branchpoint adenosine residue of the intron join during the splicing process, or
the loop portion hybridizes to a segment of the pre-splicing RNA molecule, and the segment of the pre-splicing RNA molecule spans at least one exon and at least one intron adjacent to the at least one exon.

Embodiment 6: The probe of Embodiment 1, wherein the length of the first stem portion or the length of the second stem portion ranges from 3 nucleotides to 10 nucleotides.

Embodiment 7: The probe of Embodiment 1, wherein, in the loop portion, the number of nucleotides complementary to the pre-splicing RNA molecule or the splicing product ranges from 10 to 30.

Embodiment 8: The probe of Embodiment 1, wherein the melting temperature of a hybridization product formed by the probe and the pre-splicing RNA molecule or the splicing product ranges from 40° C. to 85° C.

Embodiment 9: The probe of Embodiment 1, wherein the nucleic acid further comprises a linker, wherein the linker is at the first end of the nucleic acid, at the second end of the nucleic acid, between the first stem portion and the loop portion, and/or between the loop portion and the second stem portion.

Embodiment 10: The probe of Embodiment 9, wherein the length of the linker ranges from 1 nucleotide to 15 nucleotides.

Embodiment 11: A method of monitoring RNA splicing, wherein the method comprises preparing a mixture comprising:
an unspliced RNA molecule; and
the probe of any one of Embodiments 1-11,
wherein a change of a fluorescence signal of the probe indicates the sequence-specific splicing of the RNA molecule.

Embodiment 12: The method of Embodiment 11, wherein the amount of fluorescence signal change is proportional to the efficiency of an individual RNA splicing event.

Embodiment 13: The method of Embodiment 11, wherein the level of the fluorescence signal of the probe corresponds to the amount of the unspliced precursor RNA molecule or the level of a splicing product that derives from that precursor RNA molecule.

Embodiment 14: The method of Embodiment 11, wherein the sequence-specific splicing of the RNA molecule is specific to a developmental process or a differentiated tissue in a subject.

Embodiment 15: The method of Embodiment 11, wherein the sequence-specific splicing of the RNA molecule is present in a subject, tissue or cell having a disease or disorder, and wherein the sequence-specific splicing of the RNA molecule is not present in a corresponding healthy subject, tissue or cell.

Embodiment 16: The method of Embodiment 15, wherein the disease or disorder comprises at least one selected from the group consisting of a neurodegenerative disease, a developmental disorder, a cardiac disorder, an autoimmune disease, a cancer, and an infectious disease.

Embodiment 17: A method of screening modulators of RNA splicing, wherein the method comprises:
preparing a first mixture comprising:
   an unspliced precursor RNA molecule; and
   the probe of any one of Embodiments 1-11,
determining a first fluorescence signal level change of the first mixture;
preparing a test mixture comprising:
   the unspliced precursor RNA molecule;
   the probe of any one of Embodiments 1-11; and
   a test compound,
determining a second fluorescence signal level change of the second mixture,
wherein a difference between the first fluorescence signal level change and the second fluorescence signal level change indicates that the test compound is a modulator of the sequence-specific splicing of the RNA molecule.

Embodiment 18: The method of Embodiment 17, wherein the method is a method of screening compounds for treating, ameliorating or preventing a disease or a disorder, wherein the sequence-specific splicing of the RNA molecule is a cause or a contributing factor to the disease or the disorder.

Embodiment 19: The method of Embodiment 16, wherein the disease or the disorder comprises at least one selected from the group consisting of a neurodegenerative disease, a developmental disorder, a cardiac disorder, an autoimmune disease, a cancer or an infectious disease.

Embodiment 20: The method of Embodiment 14, wherein the method is a method of screening anti-pathogenic compounds, wherein the splicing of the RNA molecule is specific to a eukaryotic pathogen and does not take place in the host of the eukaryotic pathogen.

Embodiment 21: The method of Embodiment 11, which further comprises applying a gene editing technology that affects an amount and/or sequence of the unspliced RNA molecule or a splicing product thereof.

Embodiment 22: The method of Embodiment 21, wherein the gene editing technology comprises at least one selected from the group consisting of an RNA interference technology, a CRISPR-Cas12 system, and a CRISPR-Cas13 system.

The disclosures of each and every patent, patent application, and publication cited herein are hereby incorporated herein by reference in their entireties.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A probe for monitoring the sequence-specific splicing of individual RNA exons
the probe comprising a nucleic acid, a first fluorescent element attached to a first end of the nucleic acid, and a second fluorescent element attached to a second end of the nucleic acid, wherein the nucleic acid comprises:
a first stem portion;
a second stem portion; and
a loop portion,
wherein one of the following applies:
the loop portion of the nucleic acid does not hybridize to a pre-splicing RNA molecule and hybridizes to a splicing product of the RNA molecule, or
the loop portion of the nucleic acid hybridizes to the pre-splicing RNA molecule and does not hybridize to the splicing product of the RNA molecule,
wherein the first stem portion and the second stem portion are at least practically complementary to each other and can form a stem structure when the loop portion is not hybridized to the pre-splicing RNA molecule or the splicing product, and
wherein the fluorescent signal of the probe when the loop portion of the nucleic acid hybridizes to the pre-splicing RNA molecule or the splicing product, and the fluorescent signal of the probe when the loop portion of the nucleic acid does not hybridize to the pre-splicing RNA molecule or the splicing product, are different.

2. The probe of claim 1, wherein the nucleic acid is RNA, DNA, or a modified nucleic acid.

3. The probe of claim 1, wherein, when the loop portion of the nucleic acid is not hybridized to the pre-splicing RNA molecule or the splicing product, the first fluorescent element and the second fluorescent element are in proximity to each other such that a fluorescent property of the first fluorescent element is affected by the second fluorescent element.

4. The probe of claim 3, wherein at least one of the following applies:
the first fluorescent element is a fluorophore and the second fluorescent element is a quencher that decreases the fluorescence intensity of the fluorophore, or
the first fluorescent element is a donor in a fluorescence resonance energy transfer (FRET) pair, and the second fluorescent element is an acceptor in the FRET pair.

5. The probe of claim 1, wherein at least one of the following applies:
the loop portion hybridizes to a segment of the splicing product of the RNA molecule, and the segment of the splicing product spans at least two consecutive exons,
the loop portion hybridizes to a segment of a lariat intronic RNA formed by the splicing, and the segment of the lariat intronic RNA comprises the point where the 5′-end and the branchpoint adenosine residue of the intron join during the splicing process, or
the loop portion hybridizes to a segment of the pre-splicing RNA molecule, and the segment of the pre-splicing RNA molecule spans at least one exon and at least one intron adjacent to the at least one exon.

6. The probe of claim 1, wherein the length of the first stem portion or the length of the second stem portion ranges from 3 nucleotides to 10 nucleotides.

7. The probe of claim 1, wherein, in the loop portion, the number of nucleotides complementary to the pre-splicing RNA molecule or the splicing product ranges from 10 to 30.

8. The probe of claim 1, wherein the melting temperature of a hybridization product formed by the probe and the pre-splicing RNA molecule or the splicing product ranges from 40° C. to 85° C.

9. The probe of claim 1, wherein the nucleic acid further comprises a linker, wherein the linker is at the first end of the nucleic acid, at the second end of the nucleic acid, between the first stem portion and the loop portion, and/or between the loop portion and the second stem portion.

10. The probe of claim 9, wherein the length of the linker ranges from 1 nucleotide to 15 nucleotides.

11. A method of monitoring RNA splicing, wherein the method comprises preparing a mixture comprising:
an unspliced RNA molecule; and
the probe of claim 1,
wherein a change of a fluorescence signal of the probe indicates the sequence-specific splicing of the RNA molecule.

12. The method of claim 11, wherein the amount of fluorescence signal change is proportional to the efficiency of an individual RNA splicing event.

13. The method of claim 11, wherein the level of the fluorescence signal of the probe corresponds to the amount of the unspliced precursor RNA molecule or the level of a splicing product that derives from that precursor RNA molecule.

14. The method of claim 11, wherein the sequence-specific splicing of the RNA molecule is specific to a developmental process or a differentiated tissue in a subject.

15. The method of claim 11, wherein the sequence-specific splicing of the RNA molecule is present in a subject, tissue or cell having a disease or disorder, and wherein the sequence-specific splicing of the RNA molecule is not present in a corresponding healthy subject, tissue or cell.

16. The method of claim 15, wherein the disease or disorder comprises at least one selected from the group consisting of a neurodegenerative disease, a developmental disorder, a cardiac disorder, an autoimmune disease, a cancer, and an infectious disease.

17. A method of screening modulators of RNA splicing, wherein the method comprises:
preparing a first mixture comprising:
an unspliced precursor RNA molecule; and
the probe of claim 1,
determining a first fluorescence signal level change of the first mixture;
preparing a test mixture comprising:
the unspliced precursor RNA molecule;
the probe of claim 1; and
a test compound,
determining a second fluorescence signal level change of the second mixture,
wherein a difference between the first fluorescence signal level change and the second fluorescence signal level change indicates that the test compound is a modulator of the sequence-specific splicing of the RNA molecule.

18. The method of claim 17, wherein the method is a method of screening compounds for treating, ameliorating or preventing a disease or a disorder, wherein the sequence-specific splicing of the RNA molecule is a cause or a contributing factor to the disease or the disorder.

19. The method of claim 16, wherein the disease or the disorder comprises at least one selected from the group consisting of a neurodegenerative disease, a developmental disorder, a cardiac disorder, an autoimmune disease, a cancer or an infectious disease.

20. The method of claim 14, wherein the method is a method of screening anti-pathogenic compounds, wherein the splicing of the RNA molecule is specific to a eukaryotic pathogen and does not take place in the host of the eukaryotic pathogen.

21. The method of claim 11, which further comprises applying a gene editing technology that affects an amount and/or sequence of the unspliced RNA molecule or a splicing product thereof.

22. The method of claim 21, wherein the gene editing technology comprises at least one selected from the group consisting of an RNA interference technology, a CRISPR-Cas12 system, and a CRISPR-Cas13 system.


SEQUENCE LISTING (3 sequences)

SEQ ID NO: 1 - RNA, 32 nt, synthetic construct
  source 1..32: mol_type = other RNA
actctaggtagacgagaagaccctatgcagct

SEQ ID NO: 2 - DNA, 24 bp, synthetic construct
  source 1..24: mol_type = other DNA
  modified_base 1: mod_base = OTHER; note = Aminomodifier C3 TFA (Am) attached to the 5'-end
  modified_base 24: mod_base = OTHER; note = Black Hole Quencher 2 (BHQ2) attached to the 3'-end
ccaggagggtcttctcgtccctgg

SEQ ID NO: 3 - DNA, 28 bp, synthetic construct
  source 1..28: mol_type = other DNA
  modified_base 1: mod_base = OTHER; note = Aminomodifier C3 TFA (Am) (Glen Research) attached to the 5'-end
  modified_base 28: mod_base = OTHER; note = Black Hole Quencher 2 (BHQ2) attached to the 3'-end
ccaggatagggtcttctcgtctacctgg